(19) United States
(12) Patent Application Publication
(10) Pub. No.: US 2026/0232183 A1
(43) Pub. Date: Aug. 13, 2026

(54) VIRTUAL REALITY BASED SCREENING TOOL FOR ASSESSING AND DEMONSTRATING OCULAR CONDITIONS

(71) Applicants: Kaitlyn Elyssa Bajek, Murrells Inlet, SC (US); Tammy Tully, Myrtle Beach, SC (US)

(72) Inventors: Kaitlyn Elyssa Bajek, Murrells Inlet, SC (US); Tammy Tully, Myrtle Beach, SC (US)

(21) Appl. No.: 19/048,046

(22) Filed: Feb. 7, 2025

(51) Int. Cl. A61B 3/032; G06F 21/31

(52) U.S. Cl. CPC A61B 3/0325; G06F 21/31

(57) ABSTRACT

A VR-based screening tool for assessing and demonstrating ocular conditions is disclosed. The present invention improves on existing screening and diagnostic practices by offering an engaging, immersive VR experience that is visually appealing and easy to understand for both children and the elderly. By simplifying the assessment process and providing clear visual demonstrations, it encourages active participation and enhances the accuracy and effectiveness of ocular evaluations.

### BACKGROUND OF THE INVENTION

The present invention addresses the challenge of accurately screening/diagnosing for ocular conditions and effectively demonstrating their impact in a way that is accessible, engaging, and more precise than traditional methods. Many individuals experience delays in identifying visual issues due to a lack of advanced screening/diagnosing tools, leading to untreated conditions that can worsen over time as well as impact the psychosocial well-being of an individual. Additionally, current conventional methods often fail to visually demonstrate these conditions, making it difficult for patients and providers to fully understand and address the issues.

Existing devices in the field of ocular screening often lack engagement, are not visually appealing, and fail to provide a child-or elderly-friendly experience. They typically use processes that are difficult for younger children or elderly individuals to follow and understand, limiting their effectiveness in these populations.

The present invention improves on existing screening and diagnostic practices by offering an engaging, immersive VR experience that is visually appealing and easy to understand for both children and the elderly. By simplifying the assessment process and providing clear visual demonstrations, it encourages active participation and enhances the accuracy and effectiveness of ocular evaluations.

### SUMMARY OF THE INVENTION

In one aspect of the present invention, there is disclosed a virtual-reality-based screening tool for assessing and demonstrating ocular conditions. The present invention improves on existing screening and diagnostic practices by offering an engaging, immersive VR experience that is visually appealing and easy to understand for both children and the elderly. By simplifying the assessment process and providing clear visual demonstrations, it encourages active participation and enhances the accuracy and effectiveness of ocular evaluations.

These and other features, aspects and advantages of the present invention will become better understood with reference to the following drawings, description and claims.

### BRIEF DESCRIPTION OF THE DRAWINGS

FIG. 1 is a schematic view of a virtual-reality-based screening tool for assessing and demonstrating ocular conditions in accordance with a preferred embodiment of the present invention; and

FIG. 2 is a flowchart view of a virtual-reality-based screening tool for assessing and demonstrating ocular conditions in accordance with a preferred embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention addresses the challenge of accurately screening/diagnosing for ocular conditions and effectively demonstrating their impact in a way that is accessible, engaging, and more precise than traditional methods. Many individuals experience delays in identifying visual issues due to a lack of advanced screening/diagnosing tools, leading to untreated conditions that can worsen over time as well as impact the psychosocial well-being of an individual. Additionally, currently available methods often fail to visually demonstrate these conditions, making it difficult for patients and providers to fully understand and address the issues. The invention claimed here solves this problem.

The VR-based screening/diagnostic tool of the present invention solves these and other problems with conventional systems and methods by providing a highly immersive and interactive platform that accurately assesses a wide range of ocular conditions. It enhances the screening process with real-time simulations, allowing patients and providers to visualize the effects of these conditions. This combination of advanced screening and visual demonstration improves early detection and understanding, leading to more effective diagnosis and treatment planning.

The present invention differs from conventional systems and methods. The VR-based screening/diagnostic tool of the present invention is unique in many respects, including, but not limited to, because it combines cutting-edge virtual reality technology with ocular screening, allowing both assessment and real-time visual demonstrations of ocular conditions. Unlike traditional methods, it offers a more immersive, accurate, and engaging way to detect and understand eye health issues.

Conventional devices often do not work well because they rely on outdated methods that can be intimidating or confusing for younger patients and the elderly. This can lead to inaccurate assessments, incomplete screenings, and a lack of motivation to engage with the process, ultimately hindering early detection and treatment of ocular conditions.

The present invention improves on existing screening and diagnostic practices by offering an engaging, immersive VR experience that is visually appealing and easy to understand for both children and the elderly. By simplifying the assessment process and providing clear visual demonstrations, it encourages active participation and enhances the accuracy and effectiveness of ocular evaluations.

Also, the VR screening and diagnostic tool for ocular conditions of the present invention can produce a variety of useful products and devices that enhance the understanding and management of eye health. Firstly, it generates a comprehensive suite of virtual assessments designed to evaluate various ocular conditions, including but not limited to visual acuity tests, binocular vision evaluations, and screening for conditions such as convergence insufficiency and diplopia. Each assessment is accompanied by real-time feedback, allowing users to track their performance and identify areas of concern.

Additionally, the present invention can create educational modules that provide in-depth information about specific ocular conditions, including symptoms, causes, and recommended management strategies. These modules can be tailored to different audiences, such as patients, caregivers, and healthcare providers, ensuring that each group receives relevant information. The tool may also produce reports summarizing test results, which can be shared with eye care professionals to facilitate informed discussions during follow-up appointments.

Moreover, the VR system of the present invention can incorporate interactive simulations that visually demonstrate the effects of various ocular conditions, providing users with a firsthand understanding of how these conditions impact vision. This feature not only aids in diagnosis but also enhances patient education and engagement. Ultimately, the invention offers a comprehensive ecosystem that not only facilitates the screening and diagnosis of ocular conditions but also empowers users with knowledge and resources for better eye health management.

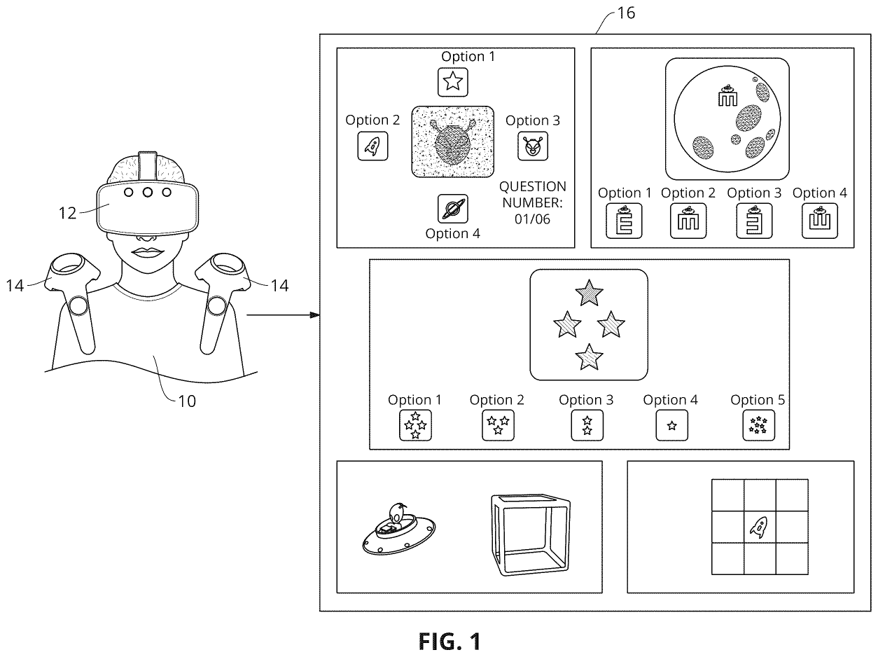

FIG. 1 depicts a schematic view of a virtual-reality-based screening tool for assessing and demonstrating ocular conditions constructed in accordance with a preferred embodiment of the present invention. FIG. 1 depicts a user 10 wearing a virtual reality headset 12 and having first and second controllers 14. An exemplary diagnostic module 16 is also shown.

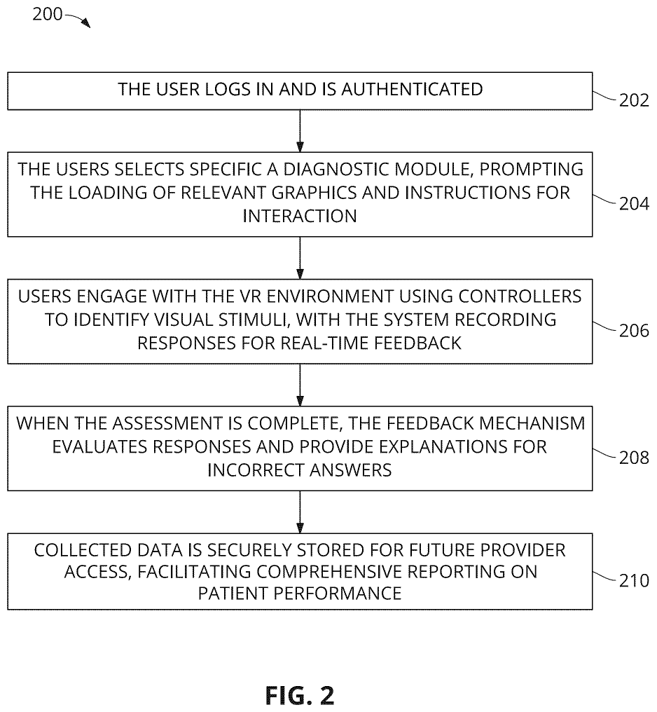

FIG. 2 depicts an exemplary flowchart 200 of the present invention. As described therein, a user would log in and be authenticated by the system at step 202. The user would then select a specific diagnostic module at step 204 which prompts the loading of relevant graphics and instructions for further interaction. The user next engages with the selected environment at step 206 by using controllers 14 to identify visual stimuli, with the system recording the responses for real-time feedback. As seen in step 208, when the assessment is complete, the feedback mechanism evaluates responses and provides explanations for incorrect answers. At step 210, the collected data is securely stored for future provider access to facilitate comprehensive reporting on the patient's performance.

The various steps discussed above are described in more detail below. In a preferred embodiment, as described herein and as seen in the accompanying Figures, the present invention comprises the following primary components/steps:
1. The VR simulation aims to provide an immersive experience that visually demonstrates various ocular conditions, helping providers, students, patients, caregivers, and parents understand the impact of these conditions on vision. This educational tool will simulate a range of ocular issues, such as color deficiency, convergence insufficiency, divergence excess, diplopia, and macular degeneration, allowing users to experience how these conditions affect vision in real-time. Users can select from a menu of ocular conditions, each accompanied by unique visual effects that mimic symptoms like blurriness, halos, dark spots, or color distortion. The simulation includes guided audio narration that educates users about each condition while they explore their environment, seeing how vision is altered in different scenarios. The environment is set up on the Meta Quest VR platform, utilizing Unity3D or Unreal Engine for development. Key assets will include graphics for each condition, audio explanations, and user interface elements for smooth navigation. Testing will ensure that simulations display accurately, provide an immersive experience, and align audio narration with visuals. This VR simulation serves as a powerful educational tool, enhancing understanding of ocular conditions and fostering empathy among all users by visually representing the challenges faced by individuals with visual impairments.
2. The game is designed to screen for color deficiency by presenting a series of color plates, allowing patients to select the symbol they see from a list of options using the Meta Quest controller. It will be developed on the Meta Quest VR platform, using either Unity3D (with C#) or Unreal Engine (with C++/Blueprints). The game features color plates with various symbols at their center, including four different options (e.g., circle, square, triangle, star) displayed on the sides of the screen. Of course, other symbols could be used as a matter of application specific design choice. The user interface will have a white background, with color plates centered prominently and multiple-choice options positioned alongside. Functionality includes loading and displaying each color plate, enabling symbol selection via the Meta Quest controller, and implementing a 20-second timer for each plate. The remaining time will not be visually displayed but will run in the background. The remaining time may also be accompanied by an audio countdown during the last five seconds (5 4 3 2 1 blast off), after which the game will automatically switch to the next color plate. The controller will vibrate upon selection to provide feedback. To initialize the game, the VR environment will be set up with a white background, and the color plates and symbols will be loaded. During the game loop, each color plate will be displayed with associated symbols, the timer will begin, and user interactions will be detected through the controller. Audio files for the countdown will be integrated to ensure they play correctly during the final moments of each display. The game needs to simulate a viewing distance of 75 cm, requiring assets such as images of color plates, icons for the symbols, and the audio files for the countdown. Thorough testing and debugging will ensure each color plate displays correctly, the timer functions smoothly, the controller vibrates upon selection, and the audio countdown plays as intended.
3. The game is designed to assess visual acuity by displaying a series of letters.
4. The game is designed to assess convergence insufficiency by requiring the patient to use red and green filters over their eyes. The patient must align a moving symbol with a moving square box and click both Meta Quest controllers when they feel the symbol is in the box, with the controllers vibrating upon clicking. Developed for the Meta Quest VR platform, the game can utilize either Unity3D (with C#) or Unreal Engine (with C++/Blueprints). Key game elements include a symbol that moves up to 6 inches (15.24 cm) to the left and a square box that moves up to 6 inches (15.24 cm) to the right, both displayed on the screen and moving simultaneously. The user interface features red and green filters, applied to the right and left eyes, respectively. Functionality involves loading and displaying the symbol and square box graphics while applying the filters. The symbol and box will move in a randomized or predetermined pattern, with the patient clicking both Meta Quest controllers when they feel the symbol is in the box. The controllers will vibrate upon clicking. The patient is required to align the symbol with the box three times during the game. To initialize the game, the VR environment will be set up with the red and green filters, and the symbol and square box graphics will be loaded. During the game loop, for each trial (three times in total), the symbol will move up to 6 inches to the left while the square box moves up to 6 inches to the right, ensuring simultaneous movement. User interactions will be detected through the Meta Quest controllers, with the controllers vibrating upon clicking. The game will check if the symbol is within the box upon clicking, resetting positions and repeating until all three trials are completed.
5. The game is designed to assess divergence excess by requiring the patient to use red and green filters over their eyes. The patient aligns a moving symbol with a moving square box and clicks both Meta Quest controllers when they feel the symbol is in the box, with the controllers vibrating upon clicking. Developed for the Meta Quest VR platform. Key game elements include a symbol that moves up to 6 inches (15.24 cm) to the right and a square box that moves up to 6 inches (15.24 cm) to the left, both displayed on the screen and moving simultaneously. The user interface features red and green filters applied to the right and left eyes, respectively. Functionality involves loading and displaying the symbol and square box graphics while applying the filters. The symbol and box will move in a randomized or predetermined pattern, and the patient will click both Meta Quest controllers when they feel the symbol is in the box. The controllers will vibrate upon clicking. The patient is required to align the symbol with the box three times during the game. To initialize the game, the VR environment will be set up with the red and green filters, and the symbol and square box graphics will be loaded. During the game loop, for each trial (three times in total), the symbol will move up to 6 inches to the right while the square box moves up to 6 inches to the left, ensuring simultaneous movement. User interactions will be detected through the Meta Quest controllers, with the controllers vibrating upon clicking. The game will check if the symbol is within the box upon clicking, resetting positions and repeating until all three trials are completed. Assets needed for the game include graphics for the symbol and square box, red and green filters for the respective eyes, and Meta Quest controller input management. Testing and debugging will ensure that the symbol and box move correctly and independently, the alignment detection logic works accurately, the controllers vibrate upon click.
6. The game is designed to assess diplopia (double vision) by requiring the patient to use a red filter over the right eye while having no filter over the left eye. The objective is for the patient to identify the position of a white rocket graphic displayed on a grid. The rocket appears in nine cardinal positions of gaze, and the patient clicks on the grid section where they perceive the rocket to be located. Developed for the Meta Quest VR platform, the game can utilize Unity3D (with C#). Key game elements include an 8 mm graphic of a white rocket and a grid representing the nine cardinal positions. The user interface features a red filter for the right eye and no filter for the left eye, along with the grid displayed on the screen. Functionality involves loading and displaying the grid and rocket graphics while applying the red filter to the right eye. The rocket appears randomly in one of the nine grid positions, accompanied by a pop sound each time it moves. Patients have 20 seconds to click on the grid section where they see the rocket, and the test runs 3 times, with the rocket appearing in different randomized positions during each trial. To initialize the game, the VR environment will be set up with the red filter for the right eye, and the grid and rocket graphics will be loaded, with the game distance set to 40 cm. During the game loop, for each trial (three times in total), the rocket will randomly be placed in one of the nine grid positions, with the pop sound played upon its appearance. User interactions will be detected through the Meta Quest controllers, which will vibrate upon clicking. The game will then check whether the patient correctly identified the rocket's position, reset, and randomize the rocket's position for the next trial.
7. The game assesses binocular vision using the Worth Four Light Test, where the patient utilizes a red filter over the right eye and a green filter over the left eye. The objective is for the patient to identify the number of stars they see through the filters at two different distances: 33 cm and 6 cm. The patient will select the number of stars perceived from multiple-choice options displayed on the side of the screen. Designed for the Meta Quest VR platform, the game can be developed using either Unity3D (with C#) or Unreal Engine (with C++/Blueprints). Key game elements include one red star at the top, two green stars on the sides, and one white star at the bottom, along with multiple-choice options for selecting the number of stars seen. The user interface features red and green filters for the respective eyes and displays the stars and multiple-choice options on the screen. Functionality includes loading and displaying the star graphics while applying the appropriate filters. The game will be played at distances of 33 cm and 6 cm. The patient has 20 seconds to click on the option corresponding to the number of stars they perceive. The test will be conducted twice-once at each distance. Of course, other symbols could be used as a matter of application specific design choice.
8. The VR screening and diagnostic tool is designed to enhance the assessment of various visual conditions through immersive simulations and can assist in therapy. By utilizing the Meta Quest VR platform and developed in environments like Unity3D or Unreal Engine, this tool provides a user-friendly interface for healthcare providers, students, and patients. The simulation covers a range of assessments, such as visual acuity tests, color vision screenings, and binocular vision evaluations. Each diagnostic test is presented in an engaging, interactive format that allows users to participate actively, making the experience both educational and effective.

### Relationship Between the Primary Components

Platform (1): The entire system operates on the Meta Quest VR platform, which provides the necessary hardware and software environment for immersive experiences.

Environment (2): The tool is developed using Unity3D or Unreal Engine, which allows for the creation of interactive and visually appealing simulations tailored for various diagnostic assessments and/or therapy.

Programming Language (3): Utilizing C#for Unity3D or C++/Blueprints for Unreal Engine ensures that the development process is efficient and allows for the integration of complex functionalities within the VR environment.

User Interface (UI) (4): The UI is designed to be intuitive and user-friendly, displaying diagnostic information, instructions, and interactive elements that guide users through the screening process.

Diagnostic Modules (5): Each module represents a specific visual assessment, such as visual acuity, color vision, or binocular vision tests. These modules are connected to the UI, allowing users to select and navigate through different assessments seamlessly.

Graphics and Visuals (6): High-quality graphics are critical for accurately simulating the visual conditions being assessed. These visuals enhance the realism of each diagnostic module and provide clear representations of how different ocular conditions appear.

Audio Feedback (7): Integrated audio feedback serves to enhance user engagement and understanding. It provides instructions, cues, and responses during the assessments, making the experience more immersive. User Interaction (8): The system utilizes interactive elements that respond to user input via the Meta Quest controllers. This interaction is crucial for conducting the assessments and ensuring that users can engage with the tool effectively.

Testing and Feedback Mechanism (9): Each module includes a built-in testing and feedback mechanism that evaluates user performance during the assessments. This component is essential for providing real-time results and guidance for both patients and providers.

Data Storage and Analysis (10): The tool can include capabilities for storing and analyzing user data, which allows healthcare providers to track progress and outcomes over time. This analysis is key for making informed decisions regarding further evaluations or treatments.

### How the Invention Works

The VR screening and diagnostic tool of the present invention operates through a series of interconnected components designed for effective assessment of ocular conditions. It starts with user authentication, ensuring only authorized individuals, providers, patients, and caregivers access the system. Once logged in, users select specific diagnostic modules, prompting the loading of relevant graphics and instructions for interaction.

During the assessment, users engage with the VR environment using controllers to identify visual stimuli, with the system recording responses for real-time feedback. After the assessment, the feedback mechanism evaluates responses, providing explanations for incorrect answers, which aids learning and understanding.

Collected data is securely stored for future provider access, facilitating comprehensive reporting on patient performance. Error handling routines ensure smooth operation, while exit logic preserves unsaved data for later resumption. Together, these components create a cohesive VR experience that assesses ocular conditions, educates users, and provides valuable insights into visual health.

To create, implement, and practice the VR screening and diagnostic tool, a structured logic framework is essential. This framework begins with the initialization logic, where, upon launching the VR platform, the necessary libraries and assets are loaded. User authentication follows, allowing providers and patients to enter their credentials. Successful authentication grants access to diagnostic modules, while failures prompt re-entry. Once in the system, users can select specific diagnostic modules, and upon selection, relevant graphics and UI elements are loaded. When an assessment starts, instructions and visuals are displayed, and user interactions such as selecting answers using controllers are recorded for immediate feedback. After completing the assessment, results are evaluated: if user inputs match expected results, a success message appears; if not, an explanation of correct answers is provided, and results are stored for future analysis.

The feedback mechanism then comes into play, offering additional resources based on performance, with options to retake tests if desired. Data management logic ensures that compiled assessment data is stored securely, allowing providers to retrieve reports while complying with privacy regulations. Error handling logic addresses any issues that arise, displaying error messages and logging for debugging, and, in critical situations, terminating the session. The exit logic ensures a smooth closure of the application by saving unsaved data. Supporting this main logic are subroutines for graphics loading, audio management, and user input handling, which streamline processes and maintain a seamless user experience. This comprehensive framework guarantees that the VR tool operates efficiently, providing an engaging and informative platform for assessing visual conditions among providers, patients, and caregivers, and can be an important and useful therapy tool.

### How to Make the Invention

To develop a VR screening and diagnostic tool for ocular conditions, the process starts with defining objectives and identifying conditions to assess, such as convergence insufficiency and strabismus. A solid understanding of established ocular testing methods like visual acuity tests and the Worth Four Light Test is crucial for a comprehensive evaluation. Knowledge of how these conditions impact daily life, including visual perception and educational performance, also informs the design.

Next, suitable VR hardware, such as Meta Quest headsets, is selected to ensure user comfort and compatibility with software development environments like Unity3D or Unreal Engine. The design focuses on creating intuitive user interfaces for healthcare providers and patients, using high-quality 3D graphics to illustrate ocular conditions and their effects.

Programming logic, using C# or C++/Blueprints, integrates features like user authentication and secure data handling, ensuring HIPAA compliance. Functionality and usability testing follow to refine interactions and establish effective feedback mechanisms.

Comprehensive training materials support implementation in clinical settings, while a pilot testing phase allows for real-world evaluations and adjustments based on user feedback. This systematic approach results in a pioneering VR diagnostic tool that enhances the screening of ocular conditions and raises awareness of their impacts on individuals' lives, grounded in established testing methodologies.

In developing a VR screening and diagnostic tool for ocular conditions, several elements are essential for its core functionality. Necessary elements include VR hardware, such as Meta Quest headsets, which deliver an immersive experience, and a software development environment like Unity3D or Unreal Engine for creating the virtual simulations. An intuitive user interface is crucial for both healthcare providers and patients to navigate the system effectively, while programming logic in languages like C# or C++ is required to integrate various features. Established ocular testing protocols, such as visual acuity tests and the Worth Four Light Test, are fundamental for ensuring accurate assessments.

Optional elements may enhance the tool's effectiveness; for example, customization features could allow users to personalize their experience by adjusting difficulty levels or visual settings. Additionally, multiplayer functionality could enable collaborative assessments between providers and patients, fostering interaction and support.

To improve the invention further, additional components could be considered, such as expanded testing modules that incorporate a broader range of ocular tests for comprehensive assessments. Implementing data analytics tools to track patient progress over time would enhance the tool's utility in clinical settings. Furthermore, including educational resources about ocular conditions could raise awareness and provide valuable support to patients and caregivers. By integrating these necessary, optional, and potential elements, the VR tool can be made more effective and beneficial in diagnosing and understanding ocular conditions.

### How To Use The Invention:

To utilize the VR screening and diagnostic tool for ocular conditions effectively, a user would follow a series of specific steps to address the problem of accurately diagnosing and understanding these conditions. The process begins with setting up the VR hardware, such as the Meta Quest headset, ensuring it is charged and calibrated. After launching the VR application designed for ocular screening, the user creates or selects a profile, entering relevant demographic information, which tailors the experience to their needs. They then navigate through a menu of available ocular tests, choosing assessments based on their symptoms or recommendations from healthcare providers.

Once engaged in the testing phase, the user dons the VR headset and follows prompts to complete selected assessments, such as identifying letters during a visual acuity test or aligning moving symbols using color filters in a binocular vision evaluation. The application provides real-time feedback on their performance, highlighting areas of concern and potential ocular conditions. After completing the tests, users can review their results, which may suggest follow-up assessments or referrals to eye care professionals. Additionally, the application offers access to educational resources about diagnosed conditions, including symptoms and management strategies, helping users and their caregivers understand the conditions better. Based on the findings, users may schedule follow-up appointments with healthcare providers, armed with insights gained from the VR tool, ultimately enhancing their overall eye health management and ensuring timely interventions.

Also, the VR screening and diagnostic tool for ocular conditions can produce a variety of useful products and devices that enhance the understanding and management of eye health. Firstly, it generates a comprehensive suite of virtual assessments designed to evaluate various ocular conditions, including visual acuity tests, binocular vision evaluations, and screening for conditions such as convergence insufficiency and diplopia. Each assessment is accompanied by real-time feedback, allowing users to track their performance and identify areas of concern.

Additionally, the invention can create educational modules that provide in-depth information about specific ocular conditions, including symptoms, causes, and recommended management strategies. These modules can be tailored to different audiences, such as patients, caregivers, and healthcare providers, ensuring that each group receives relevant information. The tool may also produce reports summarizing test results, which can be shared with eye care professionals to facilitate informed discussions during follow-up appointments and can be a useful tool during therapy.

Moreover, the VR system can incorporate interactive simulations that visually demonstrate the effects of various ocular conditions, providing users with a firsthand understanding of how these conditions impact vision. This feature not only aids in diagnosis but also enhances patient education and engagement. Ultimately, the invention offers a comprehensive ecosystem that not only facilitates the screening and diagnosis of ocular conditions but also empowers users with knowledge and resources for better eye health management.

It should be understood, of course, that the foregoing relates to exemplary embodiments of the invention and that modifications may be made without departing from the spirit and scope of the invention as set forth in the following claims.

What is claimed is:

1. A virtual-reality-based screening system for assessing and demonstrating ocular conditions, the system comprising a user wearing a virtual reality headset and having first and second controllers, the headset displaying a diagnostic module to the user,
wherein the user first logs in and is authenticated by the system,
and wherein the user next selects a diagnostic module which prompts the system to load relevant graphics and instructions for further interaction;
wherein the user engages with the selected environment by using the first and second controllers to identify visual stimuli, with the system recording the responses for real-time feedback.

2. The system of claim 1, wherein when the assessment is complete, the feedback is evaluated explanations are provided for incorrect answers.

3. The system of claim 1, wherein the collected response data is securely stored for future provider access to facilitate comprehensive reporting on the user's performance.

4. The system of claim 1, wherein the relevant graphics are designed to assess color deficiency by displaying a series of symbols including a circle, a square, a triangle and a star.

5. The system of claim 1, wherein the relevant graphics are designed to assess visual acuity by displaying a series of letters.

6. The system of claim 1, wherein the relevant graphics are designed to assess convergence insufficiency by requiring the user to use red and green filters over their eyes.

7. The system of claim 1, wherein the relevant graphics are designed to assess diplopia (double vision) by requiring the patient to use a red filter over the right eye while having no filter over the left eye.

8. The system of claim 1, wherein the relevant graphics are designed to assess binocular vision using the Worth Four Light Test, where the patient utilizes a red filter over the right eye and a green filter over the left eye.

9. The system of claim 1, wherein the relevant graphics are designed to enhance the assessment of various visual conditions through immersive simulations, such as visual acuity tests, color vision screenings, and binocular vision evaluations.

10. A method for assessing and demonstrating color deficiency of a patient by using a virtual-reality-based screening system, the method comprising:
presenting a series of color plates having various symbols at their center, including a circle, a square, a triangle and a star displayed on the sides of the screen;
enabling symbol selection via a virtual reality controller;
implementing a 20-second timer for each plate with the remaining time not being visually displayed, but may be accompanied by an audio countdown during the last five seconds, after which the game will automatically switch to the next color plate;
wherein the controller will vibrate upon selection to provide feedback wherein the game simulates a viewing distance of 75 cm.
